(19) United States
(12) Patent Application Publication
(10) Pub. No.: US 2008/0032397 A1
(43) Pub. Date: Feb. 7, 2008

(54) Substructure For Cultivating Cells And Its Use

(75) Inventor: Juha Korpinen, Nokia (FI)

Correspondence Address: VENABLE LLP, P.O. BOX 34385, WASHINGTON, DC, 20043-9998, US

(73) Assignee: Chip-Man Technologies Oy, Tampere (FI)

(21) Appl. No.: 11/628,983

(22) PCT Filed: Jul. 8, 2005
(86) PCT No.: PCT/FI05/50277

§ 371 Date: Dec. 8, 2006

(30) Foreign Application Priority Data
Jul. 9, 2004 (FI) 20040971

(51) Int. Cl. C12M 3/00; G06K 9/00

(52) U.S. Cl. 435/303.1; 382/133

(57) ABSTRACT

A substructure for cultivating cells including well-like culture sites separate from each other. The cultivation substructure is closed from above hermetically with a lid which, together with the substructure, limits an air space being common to the culture sites and having a gas inlet and a gas outlet. Water has been added onto the bottom of the cultivation substructure in the areas outside the culture sites, to maintain humidity in the air space.

The invention relates to a substructure for cultivating cells, comprising well-like culture sites separate from each other. The invention relates particularly to a so-called well plate. The invention also relates to the use of the cultivation substructure.

Cell cultures are generally used e.g. in various cytobiological and biomedical analyses. Typically, the cell material to be analyzed is cultured in a Petri dish or on a well plate placed in suitable conditions with respect to the temperature, ambient gas and illumination. At various stages of the analyses, the samples are subjected to, for example, microscopy, and in arrangements of prior art, the well plate is arranged to be examined with a microscope which may be equipped with a camera. In many studies, the same samples are examined at regular intervals so that the development of the cell can be monitored.

From patent documents, a number of apparatuses are known which have been constructed in such a way that cells to be cultured in a sub-structure, for example in a well plate, can be kept in desired culturing environment (temperature, culture medium, atmosphere). To monitor the development of cells, for example their growth and/or the way in which they are affected by given substances added in the culture medium, it is necessary to take images at different sites of the cultivation substructure in such a way that the same sites (for example, separate wells) are imaged at regular intervals according to a given schedule.

In the simplest form, the imaging is carried out by taking the cultivation substructure out of the culturing chamber (e.g. incubator) and placing it onto a particular microscope base where the imaging can be carried out automatically, manipulators moving the base with respect to the objective of the microscope. The images thus taken are recorded in a memory and they can be processed later on. In a more automated version, the cultivation substructures are automatically removed from the incubator to be imaged according to a predetermined program. An apparatus for implementing this is presented e.g. in U.S. Pat. No. 5,106,584.

The removal of the cultivation substructures from the incubator for imaging may be a disturbing factor, because the cultivation substructure with the cells is removed from the environment where the conditions have been adjusted to be optimal, and it may also be subjected to bumps when it is being moved from one base to another. Therefore, apparatuses are also known for carrying out the imaging when the cultivation substructure is in the incubator. For example, U.S. Pat. No. 6,271,022 discloses an apparatus in which the imaging is arranged inside the incubator in such a way that substructures placed on shelves on top of each other can be imaged one after the other, although accurate imaging is not achieved by this method.

U.S. Pat. No. 6,008,010 presents an apparatus in which a well plate is placed on top of the transparent bottom of a closed incubator chamber. The cover of the incubator chamber is transparent as well, wherein imaging can be performed in the vertical direction through the incubator chamber by using a manipulator to move the whole chamber in the horizontal direction in relation to the imaging optics. With this system, it is already possible to perform imaging in situ without removing the cultivation substructure from the chamber. However, a problem lies in the fact that the whole culture chamber must be moved. Because of this, the culture chamber, or “biochamber”, has been made relatively small (length×width×height 6″×5″×2″). Moreover, the use of a transparent extra plate under the bottom of the well plate is problematic for high magnifications, because the objective must be brought very close to the object.

Consequently, it is known to place well plates in an incubator having desired ambient conditions (humidity, temperature, gas composition). For example, an incubator chamber according to U.S. Pat. No. 6,271,022 containing several cultivation substructures in shelves is arranged to be air-tight to provide a given CO₂ level inside the chamber. It is difficult to provide such uniform conditions for all the cultivation substructures. One problem is, for example, the control of humidity. Furthermore, as the cultivation substructure is removed from the incubator, it is exposed to external conditions, which may be harmful if the cultivation substructure must be removed for any reason.

It is an aim of the invention to eliminate the above-mentioned drawbacks and to present a cultivation substructure which constitutes a compact unit and in which the desired conditions can be maintained better. To achieve this aim, the cultivation substructure according to the invention is primarily characterized in that the cultivation substructure is closed hermetically from above with a lid which, together with the substructure, limits an air space being common to the culture sites and having a gas inlet and a gas outlet, and that water has been added onto the bottom of the cultivation substructure in the areas outside the culture sites, to maintain humidity in the air space. The humidity prevents the drying of the wells.

In this way, a kind of a mini incubator can be formed of the cultivation substructure by connecting to it a lid with an inlet and an outlet provided ready for a gas. Via the inlet, it is possible to supply a desired gas composition, for example a composition with a desired content of oxygen and/or carbon dioxide. Most commonly, the carbon dioxide content of 5% is used in the air to be supplied. The outlet can be connected to a check valve letting gas through in one direction only, away from the air space. It is possible to use a normal well plate which can be easily converted to said mini incubator by means of the invention.

With respect to the maintenance of the gas composition and humidity, the mini incubator can be placed as a separate, independent unit in a larger incubator chamber. To image cells on the cultivation substructure in the incubator environment at given intervals, the imaging optics and the unit can be moved in relation to each other in the incubator chamber, wherein the different culture sites (wells) are imaged.

In the following, the invention will be described in more detail with reference to the appended drawings, in which

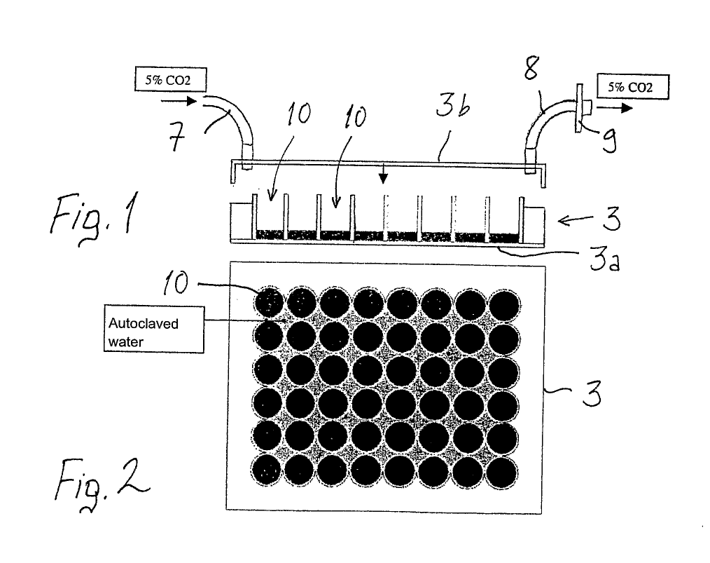

FIG. 1 shows a cultivation substructure seen from the side,

FIG. 2 shows the cultivation substructure seen from above,

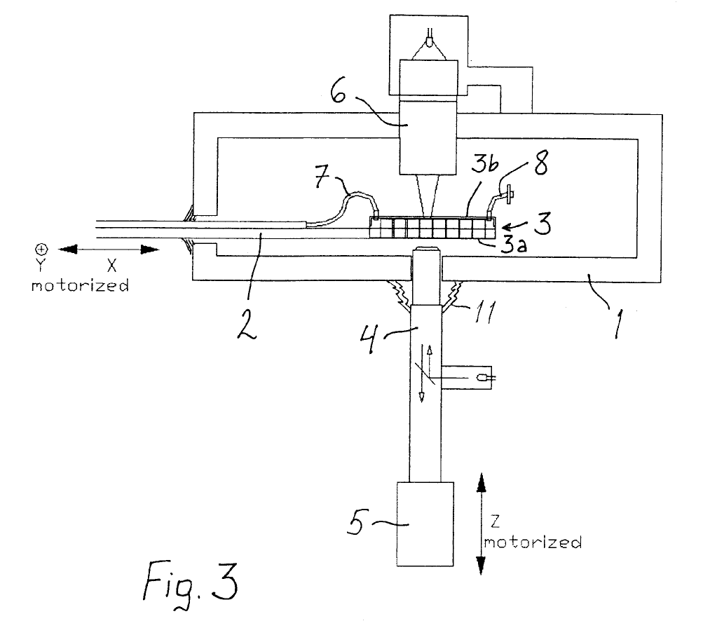

FIG. 3 shows the use of the cultivation substructure in a larger incubator chamber,

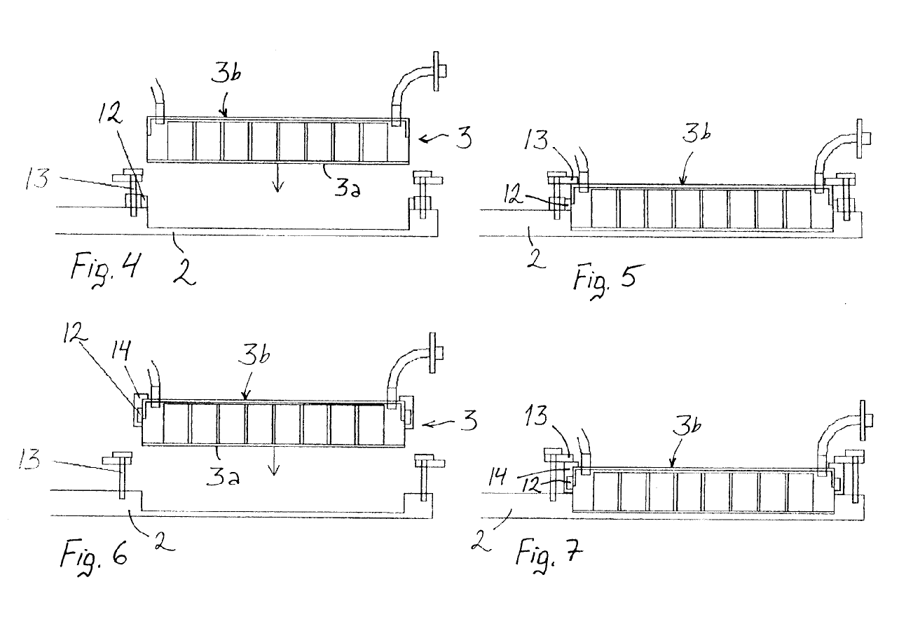

FIGS. 4 and 5 show one option of fixing the cultivation substructure, and

FIGS. 6 and 7 show another option of fixing the cultivation substructure.

FIG. 1 shows a well plate 3 which is used as a cultivation substructure and which comprises wells 10 next to each other in the plane of the substructure, the wells constituting separate culture sites for culturing cells. The wells contain a culture medium whose composition depends on the cell to be cultured. The wells are normally arranged as a n×m matrix in the plane of the plate, i.e. in n rows and m columns. FIG. 2 shows a 6×8 plate, i.e. a plate of 48 wells, but also other types of plates can be used.

The well plate 3 is closed from above with a lid 3b in an air-tight manner. The lid 3b comprises downwards extending edges (a flange) by means of which the lid can be fixed to the plate by pressing, and by sealing the joint it is possible to produce a mini incubator isolated from the environment. The lid is also provided with an inlet and an outlet, through which a gas can be introduced into the closed air space underneath the lid 3b, and be removed from it, respectively. The gas inlet and outlet ducts are marked with reference numerals 7 and 8, respectively. The outlet duct 8 comprises a valve 9 which prevents an air flow from the environment into the mini incubator but lets gas out. A pump or a gas bottle and adjustable valves, are provided at the initial end of the inlet duct 7 to supply a gas with a desired composition at intervals into the mini incubator.

Generally, the gas is air with a carbon dioxide content higher than in normal air, to buffer the culture medium in the wells 10. Normally, the content of 5% is used. It is possible that instead of or in addition to carbon dioxide, the content of another gas component should be set to a desired level, for example the oxygen content.

Water has been added onto the bottom of the well plate in the space between the wells. The purpose of this water is to keep the air in the mini incubator humid. Consequently, drying of the wells is avoided, and it is not necessary to control the humidity of the air to be supplied. FIG. 1 illustrates the water level in the space outside the wells 10. A requirement for the use of water is that the well plate has such a structure in which the surface (bottom) of the plate outside the wells 10 lies lower than the upper edges of the wells 10.

Both the bottom 3a and the lid 3b of the well plate are made of a transparent material, for example optically clear plastic, wherein microscopic imaging in the vertical direction through the well plate is possible.

The temperature of the mini incubator can be adjusted by placing it in a larger chamber whose temperature is controlled.

FIG. 3 shows one possibility for using the mini incubator of a well plate and a lid in a culture apparatus. The well plate closed with the lid, i.e. the mini incubator 3, is arranged movable in the X-Y plane by means of a moving manipulator arm 2 inside a culture chamber 1 with a controlled temperature. In the figure, reference numeral 4 indicates a microscope, which is arranged to be movable, together with a digital camera 5, by means of a motor in direction Z perpendicular to the X-Y plane of moving of the cultivation substructure. Reference numeral 6 indicates an illuminator device to provide illumination from the opposite direction (from above). The microscope 4 is a tube microscope which is focused by moving the microscope in the Z direction.

The culture chamber 1 constitutes a dark chamber protected from ambient light, and its temperature can also be adjusted to a desired level irrespective of ambient temperature. The arm 2 is introduced via a through-hole in the side of the chamber, sealed with e.g. elastic means so that no light or heat can enter through the hole but the arm 2 can move in the hole. Similarly, the microscope 4 is introduced into the culture chamber 1 via a sealed opening (sealing ring 11).

According to the above-mentioned principle, it is possible to provide the mini incubator 3 with a desired gas composition, irrespective of the gas composition inside the culture chamber 1.

Imaging is performed in the normal way, one well 10 of the well plate at a time, wherein several sites of a single well can be imaged. The well 10 of the well plate to be imaged is determined by means of the manipulator moving the mini incubator 3 in the X-Y plane. Furthermore, in each well it is possible to take images from different sites in the Z direction by moving the microscope in a direction perpendicular to the X,Y plane, for example to take a series of images of the same well in different focusing planes. During the imaging, the illumination can be provided by using illumination whose duration is set accurately with a system presented in more detail in a parallel patent application “An illumination system for a microscope” filed simultaneously.

The alternatives relating to the storage and processing of the image itself do not fall within the scope of the present invention, and they will thus not be disclosed in more detail.

However, the invention can also be applied elsewhere than in the environment shown in FIG. 3.

FIGS. 4 and 5 show a first way how the lid 3b is fixed in an air-tight manner to the well plate, and a mini incubator formed by them is fixed to the manipulator arm 2. The manipulator arm comprises an opening or a “fixing window” to which the mini incubator can be fixed in such a way that the bottom 3a is not covered but can be imaged directly from below through the opening. Here an insulation 12 for sealing the joint between the lid 3b and the well plate is placed around the fixing opening to form a kind of a sealing frame. Mechanical fixing means 13 are provided above the insulation 12, and they can be turned onto the lid 3b after the lid 3b and the well plate have been placed in the opening (FIG. 5).

FIGS. 6 and 7 show a fixing principle which differs from the above one in that the insulation 12 around the well plate is placed at the joint between the lid 3b and the well plate in the mini incubator to be ready before the mini incubator is placed into the opening. The keeping of the insulation 12 in its position is further secured with a holder 14.

1-6. (canceled)

7. A substructure for cultivating cells, comprising:
well-like culture sites separate from each other and defining upper edges;
a bottom;
a lid closing said culture sites from above, said lid limiting an air space common to the culture sites and closing said cultivation substructure hermetically to provide a mini incubator isolated from the environment;
provided in the lid, a gas inlet arranged to supply a desired gas composition through an inlet duct;
a gas outlet provided in the lid; and
water on a bottom in a space between the culture sites where the bottom lies lower than the upper edges of the well-like culture sites, to maintain humidity in said air space common to the culture sites.

8. The cultivation substructure according to claim 7, wherein the inlet is connected by the inlet duct to a source of gas with a desired controlled composition.

9. The cultivation substructure according to claim 8, wherein carbon dioxide content of the gas source has been adjusted to be higher than the normal carbon dioxide content of air.

10. The cultivation substructure according to claim 9, wherein the carbon dioxide content has been adjusted to a level of about 5%.

11. The cultivation substructure according to claim 7, wherein the outlet is connected to a valve letting gas flow away from the air space only.

12. The cultivation substructure according to claim 8, wherein the outlet is connected to a valve letting gas flow away from the air space only.

13. The cultivation substructure according to claim 9, wherein the outlet is connected to a valve letting gas flow away from the air space only.

14. The cultivation substructure according to claim 7, wherein the lid closes the cultivation substructure along a joint that is sealed with an insulation.

15. The cultivation substructure according to claim 8, wherein the lid closes the cultivation substructure along a joint that is sealed with an insulation.

16. The cultivation substructure according to claim 9, wherein the lid closes the cultivation substructure along a joint that is sealed with an insulation.

17. The use of a cultivation substructure in an apparatus for imaging cells, said cultivation substructure comprising:
well-like culture sites separate from each other and defining upper edges;
a bottom;
a lid closing said culture sites from above, said lid limiting an air space common to the culture sites and closing said cultivation substructure hermetically to provide a mini incubator isolated from the environment;
provided in the lid, a gas inlet arranged to supply a desired gas composition through an inlet duct;
a gas outlet provided in the lid; and
said cultivation substructure closed with the lid being placed in a culture chamber encompassing the cultivation substructure, said apparatus for imaging cells further comprising imaging optics, as well as actuators for providing the relative movement of the cultivation sub-structure and the imaging optics in such a way that the imaging optics is used to image different sites of the cultivation substructure by moving the imaging optics and the cultivation substructure in relation to each other.

18. The use according to claim 17, wherein gas with a desired controlled composition is supplied to the cultivation substructure by the inlet duct.

19. The use according to claim 18, wherein the gas supplied has carbon dioxide content higher than the normal carbon dioxide content of air.

20. The use according to claim 17, wherein said cultivation substructure contains water on its bottom in the space between the culture sites where the bottom lies lower than the upper edges of the well-like culture sites, and the water is used to maintain humidity in said air space common to the culture sites.

21. The use according to claim 18, wherein said cultivation substructure contains water on its bottom in the space between the culture sites where the bottom lies lower than the upper edges of the well-like culture sites, and the water is used to maintain humidity in said air space common to the culture sites.

22. The use according to claim 19, wherein said cultivation substructure contains water on its bottom in the space between the culture sites where the bottom lies lower than the upper edges of the well-like culture sites, and the water is used to maintain humidity in said air space common to the culture sites.
